SEQUENCE LISTING (3 sequences)

SEQ ID NO: 1 - RNA, 20 nt, SARS-CoV-2
  source 1..20: mol_type = other RNA
atgctgcaatcgtgctacaa

SEQ ID NO: 2 - RNA, 17 nt, SARS-CoV-2
  source 1..17: mol_type = other RNA
gactgccgcctctgctc

SEQ ID NO: 3 - RNA, 20 nt, SARS-CoV-2
  source 1..20: mol_type = other RNA
tcaaggaacaacattgccaa

(19) United States
(12) Patent Application Publication
(10) Pub. No.: US 2025/0009713 A1
(43) Pub. Date: Jan. 9, 2025

(54) METHODS OF TREATING SARS-COV-2 INFECTION

(71) Applicant: BIOTRON LIMITED, Sydney, New South Wales (AU)

(72) Inventors: Michelle MILLER, Sydney (AU); Gary Dinneen EWART, Sydney (AU)

(73) Assignee: BIOTRON LIMITED, Sydney, New South Wales (AU)

(21) Appl. No.: 18/712,518

(22) PCT Filed: Nov. 24, 2022
(86) PCT No.: PCT/AU2022/051405

§ 371 Date: May 22, 2024

(30) Foreign Application Priority Data
Nov. 24, 2021 (AU) 2021903789

(51) Int. Cl. A61K 31/415; A61P 31/14

(52) U.S. Cl. CPC A61K 31/415; A61P 31/14

(57) ABSTRACT

The present invention relates to the treatment or prevention of SARS-COV-2 infection or COVID-19. In particular, the present invention relates to the use of N-carbamimidoyl-5-(1-methyl-1H-pyrazol-4-yl)-2-naphthamide, or a pharmaceutically acceptable salt thereof, in the treatment or prevention of SARS-COV-2 infection or COVID-19.

### FIELD OF THE INVENTION

The present application claims priority from Australian Provisional Patent Application No. 2021903789 (filed 24 Nov. 2021), the contents of which are incorporated in their entirety herein.

The present invention relates to the treatment or prevention of SARS-COV-2 infection. In particular, the present invention relates to antiviral compounds and their use in the treatment or prevention of SARS-COV-2 infection.

### BACKGROUND OF THE INVENTION

Any discussion of the prior art throughout the specification should in no way be considered as an admission that such prior art is widely known or forms part of common general knowledge in the field.

Coronaviruses are a large family of viruses that cause illness ranging from the common cold to more severe diseases such as Middle East Respiratory Syndrome (MERS) and Severe Acute Respiratory Syndrome (SARS). Severe Acute Respiratory Syndrome coronavirus 2 (SARS-COV-2) is a new strain of coronavirus that is airborne, highly contagious and causes disease referred to as COVID-19. COVID-19 was declared a pandemic by the World Health Organisation on 11 Mar. 2020.

The clinical spectrum of COVID-19 ranges from mild, self-limiting respiratory tract illness to severe progressive pneumonia. In addition, many people do not fully recover from the initial respiratory illness and go on to suffer from post-COVID-19 syndrome referred to as long-COVID. The most common symptoms of long-COVID are fatigue, shortness of breath, tightness of the chest, racing heart, difficulty concentrating and brain fog, loss of smell and taste, loss of appetite, hair loss, difficulty sleeping, anxiety and depression (Huang et al. 2021, Lancet 397, 220-232). Many of those who suffer from long-COVID had mild initial symptoms and were not hospitalised. The effects of long-COVID are currently associated with a chronic aberrant immune response (Paull et al. 2021, Viruses, 13:1656).

There is an urgent need to develop strategies to prevent SARS-COV-2 infection to eliminate the potential adverse events that infection can cause.

It is an object of the present invention to overcome or ameliorate at least one of the disadvantages of the prior art, or to provide a useful alternative.

### SUMMARY OF THE INVENTION

The present application surprisingly shows that N-carbamimidoyl-5-(1-methyl-1H-pyrazol-4-yl)-2-naphthamide (BIT225) inhibits SARS-COV-2 replication, reduces infectious viral load and reduces the production of pro-inflammatory cytokines and chemokines.

The chemical structure of BIT225 is shown below:

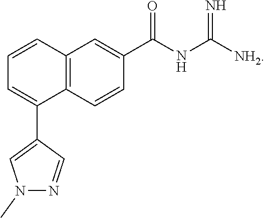 [SMILES: Cn1cc(-c2cccc3cc(C(=O)NC(=N)N)ccc23)cn1]

BIT225 may also be referred to as N-carbamimidoyl-5-(1-methylpyrazol-4-yl) naphthalene-2-carboxamide or 5-(1-methylpyrazol-4-yl) 2-naphthoylguanidine.

The present invention generally relates to the use of BIT225, or a pharmaceutically acceptable salt thereof, for treating or preventing SARS-COV-2 infection, for inhibiting the replication of SARS-COV-2, for reducing the severity, intensity, or duration of complications or symptoms associated with SARS-COV-2 infection, for reducing infectious viral load in a subject infected with SARS-COV-2, or for reducing the production of pro-inflammatory cytokines and chemokines in a subject infected with SARS-COV-2.

According to one aspect, the present invention provides a method for the treatment or prevention of SARS-COV-2 infection in a subject, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method for inhibiting the replication of SARS-COV-2 in a subject, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method for reducing the severity, intensity, or duration of complications or symptoms associated with SARS-COV-2 infection in a subject, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method of reducing viral load in a subject infected with SARS-COV-2, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method of reducing the production of a proinflammatory cytokine or chemokine in a subject infected with SARS-CoV-2, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method of reducing the concentration of a proinflammatory cytokine or chemokine in a subject infected with SARS-COV-2, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method of reducing the concentration of a proinflammatory cytokine or chemokine in the lung or serum of a subject infected with SARS-COV-2, the method comprising administering to the subject an effective amount of N-carbamimidoyl-5-(1-methyl-1H-pyrazol-4-yl)-2-naphthamide, or a pharmaceutically acceptable salt thereof.

According to one aspect, the present invention provides a method for the treatment or prevention of COVID-19 in a subject, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides a method for reducing the severity, intensity, or duration of complications or symptoms associated with COVID-19 in a subject, the method comprising administering to the subject an effective amount of N-carbamimidoyl-5-(1-methyl-1H-pyrazol-4-yl)-2-naphthamide, or a pharmaceutically acceptable salt thereof.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for the treatment or prevention of SARS-COV-2 infection.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for inhibiting the replication of SARS-COV-2.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for reducing the severity, intensity, or duration of complications or symptoms associated with SARS-COV-2 infection.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for reducing viral load in a subject infected with SARS-COV-2.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for reducing the production of a proinflammatory cytokine or chemokine in a subject infected with SARS-COV-2.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for reducing the concentration of a proinflammatory cytokine or chemokine in a subject infected with SARS-COV-2.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for reducing the concentration of a proinflammatory cytokine or chemokine in the lung or serum of a subject infected with SARS-COV-2.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for the treatment or prevention of COVID-19.

According to another aspect, the present invention provides use of N-carbamimidoyl-5-(1-methyl-1H-pyrazol-4-yl)-2-naphthamide, or a pharmaceutically acceptable salt thereof, for the manufacture of a medicament for reducing the severity, intensity, or duration of complications or symptoms associated with COVID-19.

According to another aspect, the present invention provides a composition comprising N-carbamimidoyl-5-(1-methyl-1H-pyrazol-4-yl)-2-naphthamide, or a pharmaceutically acceptable salt thereof, for use in a method of treating or preventing SARS-CoV-2 infection.

According to another aspect, the present invention provides a composition comprising N-carbamimidoyl-5-(1-methyl-1H-pyrazol-4-yl)-2-naphthamide, or a pharmaceutically acceptable salt thereof, for use in a method of inhibiting the replication of SARS-COV-2.

According to another aspect, the present invention provides a composition comprising N-carbamimidoyl-5-(1-methyl-1H-pyrazol-4-yl)-2-naphthamide, or a pharmaceutically acceptable salt thereof, for use in a method of reducing the severity, intensity, or duration of complications or symptoms associated with SARS-COV-2 infection.

According to another aspect, the present invention provides a composition comprising N-carbamimidoyl-5-(1-methyl-1H-pyrazol-4-yl)-2-naphthamide, or a pharmaceutically acceptable salt thereof, for use in a method of reducing viral load in a subject infected with SARS-COV-2.

According to another aspect, the present invention provides a composition comprising N-carbamimidoyl-5-(1-methyl-1H-pyrazol-4-yl)-2-naphthamide, or a pharmaceutically acceptable salt thereof, for use in a method of reducing the production of pro-inflammatory cytokines and chemokines in a subject infected with SARS-COV-2.

According to another aspect, the present invention provides a composition comprising N-carbamimidoyl-5-(1-methyl-1H-pyrazol-4-yl)-2-naphthamide, or a pharmaceutically acceptable salt thereof, for use in a method of treating or preventing COVID-19.

According to another aspect, the present invention provides a composition comprising N-carbamimidoyl-5-(1-methyl-1H-pyrazol-4-yl)-2-naphthamide, or a pharmaceutically acceptable salt thereof, for use in a method of reducing the severity, intensity, or duration of complications or symptoms associated with COVID-19.

According to another aspect, the present invention provides a composition comprising N-carbamimidoyl-5-(1-methyl-1H-pyrazol-4-yl)-2-naphthamide, or a pharmaceutically acceptable salt thereof, for use in reducing the production of a proinflammatory cytokine or chemokine in a subject infected with SARS-COV-2.

According to another aspect, the present invention provides a composition comprising N-carbamimidoyl-5-(1-methyl-1H-pyrazol-4-yl)-2-naphthamide, or a pharmaceutically acceptable salt thereof, for use in reducing the concentration of a proinflammatory cytokine or chemokine in a subject infected with SARS-COV-2.

According to another aspect, the present invention provides a composition comprising N-carbamimidoyl-5-(1-methyl-1H-pyrazol-4-yl)-2-naphthamide, or a pharmaceutically acceptable salt thereof, for use in reducing the concentration of a proinflammatory cytokine or chemokine in the lung or serum of a subject infected with SARS-CoV-2.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for treating or preventing SARS-COV-2 infection.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for inhibiting the replication of SARS-COV-2.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing the severity, intensity, or duration of complications or symptoms associated with SARS-COV-2 infection.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing viral load in a subject infected with SARS-COV-2.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing the production of pro-inflammatory cytokines and chemokines in a subject infected with SARS-COV-2.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for treating or preventing COVID-19.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing the severity, intensity, or duration of complications or symptoms associated with COVID-19.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing the production of a proinflammatory cytokine or chemokine in a subject infected with SARS-COV-2.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing the concentration of a proinflammatory cytokine or chemokine in a subject infected with SARS-COV-2.

According to another aspect, the present invention provides a pharmaceutical composition comprising N-carbamimidoyl-5-(1-methyl-1H-pyrazol-4-yl)-2-naphthamide, or a pharmaceutically acceptable salt thereof, for reducing the concentration of a proinflammatory cytokine or chemokine in the lung or serum of a subject infected with SARS-COV-2.

In certain embodiments, the SARS-COV-2 is a strain selected from the group consisting of US-WA1/2020 (NR-52281), US-PHC658/2021 (delta variant; NR-55611), SouthAfrica/KRISP-K005325/2020 (beta variant; NR-54009), England/204820464/2020 (alpha variant; NR-54000), Japan/TY7-503/2021-Brazil_P.1 (NR-54982) and USA/MD-HP20874/2021 (omicron variant; NR-56461).

In certain embodiments, the proinflammatory cytokine or chemokine is selected from the group consisting of Interleukin 6 (IL-6), Interleukin 1-alpha (IL-1 alpha), Tumour Necrosis Factor alpha (TNF alpha), Transforming growth factor beta (TGF beta), Monocyte Chemoattractant Protein-1 (MCP 1) and Interleukin 1 beta (IL-1 beta).

In certain embodiments, the N-carbamimidoyl-5-(1-methyl-1H-pyrazol-4-yl)-2-naphthamide, or a pharmaceutically acceptable salt thereof, is administered by a route selected from oral, nasal, intravenous, intraperitoneal, inhalation and topical.

In certain embodiments, the N-carbamimidoyl-5-(1-methyl-1H-pyrazol-4-yl)-2-naphthamide, or a pharmaceutically acceptable salt thereof, is administered orally.

In certain embodiments, the N-carbamimidoyl-5-(1-methyl-1H-pyrazol-4-yl)-2-naphthamide, or a pharmaceutically acceptable salt thereof, is administered daily.

In certain embodiments, the N-carbamimidoyl-5-(1-methyl-1H-pyrazol-4-yl)-2-naphthamide, or a pharmaceutically acceptable salt thereof, is administered twice daily.

In certain embodiments, the N-carbamimidoyl-5-(1-methyl-1H-pyrazol-4-yl)-2-naphthamide, or a pharmaceutically acceptable salt thereof, is administered at a dosage of about 100 mg to about 600 mg.

In certain embodiments, the N-carbamimidoyl-5-(1-methyl-1H-pyrazol-4-yl)-2-naphthamide, or a pharmaceutically acceptable salt thereof, is administered orally and is administered at a dosage of about 600 mg.

In certain embodiments, the N-carbamimidoyl-5-(1-methyl-1H-pyrazol-4-yl)-2-naphthamide, or a pharmaceutically acceptable salt thereof, is administered orally and is administered at a dosage of about 200 mg.

In certain embodiments, the N-carbamimidoyl-5-(1-methyl-1H-pyrazol-4-yl)-2-naphthamide, or a pharmaceutically acceptable salt thereof, is administered orally and is administered at a dosage of about 100 mg.

In certain embodiments, the N-carbamimidoyl-5-(1-methyl-1H-pyrazol-4-yl)-2-naphthamide, or a pharmaceutically acceptable salt thereof, is administered orally and is administered daily.

In certain embodiments, the N-carbamimidoyl-5-(1-methyl-1H-pyrazol-4-yl)-2-naphthamide, or a pharmaceutically acceptable salt thereof, is administered orally and is administered twice daily.

In certain embodiments, the N-carbamimidoyl-5-(1-methyl-1H-pyrazol-4-yl)-2-naphthamide, or a pharmaceutically acceptable salt thereof, is administered orally, once daily at a dosage of about 200 mg.

In certain embodiments, the N-carbamimidoyl-5-(1-methyl-1H-pyrazol-4-yl)-2-naphthamide, or a pharmaceutically acceptable salt thereof, is administered orally, twice daily at a dosage of about 200 mg.

In certain embodiments, the N-carbamimidoyl-5-(1-methyl-1H-pyrazol-4-yl)-2-naphthamide, or a pharmaceutically acceptable salt thereof, is administered orally, once daily at a dosage of about 100 mg.

In certain embodiments, the N-carbamimidoyl-5-(1-methyl-1H-pyrazol-4-yl)-2-naphthamide, or a pharmaceutically acceptable salt thereof, is administered orally, twice daily at a dosage of about 100 mg.

In certain embodiments, the N-carbamimidoyl-5-(1-methyl-1H-pyrazol-4-yl)-2-naphthamide, or a pharmaceutically acceptable salt thereof, is administered in combination with one or more additional antiviral agents.

### BRIEF DESCRIPTION OF THE DRAWINGS

FIG. 1: BIT225 dose response curves for viral loads assessed by qPCR in monolayers of Vero E6 cells or Calu-3 cells in triplicate wells pre-exposed to one of 6 concentrations of BIT225 for 1 hour, then infected (m.o.i=0.1) with one of 6 SARS-COV-2 strains: “BR” (triangles-Japan/TY7-503/2021-Brazil_P.1), “Delta” (“+” symbols-US-PHC658/2021), “omicron” (“x” symbols-USA/MD-HP20874/2021), “SA” (diamonds-SouthAfrica/KRISP-K005325/2020), “UK” (inverted triangles-England/204820464/2020), and US (filled circles-US-WA1/2020).

FIG. 2: Summary of EC50 values for viral loads assessed by qPCR derived from both cells types (error bars are 95% asymptotic-based confidence intervals on the EC50 estimates).

FIG. 3: BIT225 dose response curves for infectious virus titre assessed by plaque assay in monolayers of Vero E6 cells or Calu-3 cells in triplicate wells pre-exposed to one of 6 concentrations of BIT225 for 1 hour, then infected (m.o.i=0.1) with one of 6 SARS-CoV-2 strains: “BR” (triangles-Japan/TY7-503/2021-Brazil_P.1), “Delta” (“+” symbols-US-PHC658/2021), “omicron” (“x” symbols-USA/MD-HP20874/2021), “SA” (diamonds-SouthAfrica/KRISP-K005325/2020), “UK” (inverted triangles-England/204820464/2020), and US (filled circles-US-WA1/2020).

FIG. 4: Summary of EC50 values for infectious virus titre assessed by plaque assay from both cells (error bars are 95% asymptotic-based confidence intervals on the EC50 estimates).

FIG. 5: Effect of BIT225 on Body Weight and Mortality in SARS-2 Infected K18-hACE2 Mice. Time courses for body-weight changes-as percent of baseline pre-infection weight-are shown for Experiment 1 (A) and Experiment 2 (B and C). Dosing with BIT225 (BID) was commenced 12 hours prior to intranasal infection with 104 PFU of SARS-COV-2 (strain WA1). In A, the group mean trendlines are shown for mice dosed (BID) with vehicle control (triangles); BIT225 (100 mg/kg-diamonds & dashed line); or BIT225 (300 mg/kg-squares & solid line). Error bars are 95% confidence intervals on the mean (n=5 per group). B shows body-weight changes associated with BIT225 (300 mg/kg) doses (squares) or vehicle (triangles) for each mouse. C; Kaplan-Meier mortality curves for mice in the 12-day study: BIT225 group (solid line) versus vehicle control (dashed line).

FIG. 6: Effect on Body Weight and Mortality for mice receiving BIT225 at different start times. In A and B, four groups (n=5) of K18-hACE2 mice were infected intranasally with 104 PFU of SARS-COV-2 (strain WA1) and dosed twice daily with BIT225 (300 mg/kg) starting 24 h pre-infection (solid squares); 24 h post-infection (open squares with “+”); 48 h post-infection (open triangles), or not at all for the vehicle group (solid triangles). C; Kaplan-Meier mortality curves for each group versus vehicle (control vehicle=dashed line; 24 h pre-infection=solid line; 24 h post-infection=dashed line with circles; 48 h post-infection=crosses). All mice treated with BIT225 survived to Day 12-except one mouse in the 48 h post-infection group that died during Day 11.

FIG. 7: Dose Responsive Viral Load Reduction in Mice Treated with BIT225 for 7 Days. Lung (A & B) and serum (C & D) samples were harvested at Day 7 and analysed for viral load (A & C) by qRT-PCR; or infectious virus titre (B & D) by plaque assay. Symbols represent data for individual mice: Vehicle control (triangles); BIT225 (100 mg/kg-diamonds); BIT225 (300 mg/kg-squares). Horizontal lines and “+” indicate the group median and mean, respectively. Welch's T-tests were used to compare the group means and P-values are indicated as: ** P<0.01; *** P<0.001.

FIG. 8: Viral Load Reduction in Mice Treated with BIT225 for 5 or 12 Days. Two groups of N=4 mice were scheduled for 5 days treatment with BIT225 or vehicle control, and two separate groups of N=7 mice were similarly scheduled for 12 days treatment. Lungs were harvested from surviving mice at Day 5 or Day 12 post infection and analysed for viral load (A) by qRT-PCR; or infectious virus titre (B) by plaque assay. Symbols represent data for individual mice: Vehicle control (triangles); BIT225 (300 mg/kg-squares). Horizontal lines and “+” indicate the group median and mean, respectively. Welch's T-tests were used to compare the Day 5 control (N=3) and BIT225 (N=4) groups and P-values are indicated as: ** P<0.01. Note that, as per FIG. 2, no mice in the control group survived until day 12, whereas all seven mice in the BIT225 group survived.

FIG. 9: Dose Responsive Cytokine Levels in Mice Treated with BIT225 for 7 Days: (A) Lung; (B) Serum. Lungs were harvested from surviving mice at Day 7 post infection and analysed for concentration of the indicated cytokines or chemokine by sandwich ELISA assay. Symbols represent data for individual mice: Vehicle control (triangles); BIT225 (100 mg/kg—diamonds); BIT225 (300 mg/kg—squares). Horizontal lines and “+” indicate the group median and mean, respectively. Welch's T-tests were used to compare the group means (N=5 mice) and P-values are indicated as: ns-P>0.05; * P<0.05; ** P<0.01; *** P<0.001.

### DEFINITIONS

In describing and claiming the present invention, the following terminology has been used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

In the context of the present invention, the words “comprise”, “comprising” and the like are to be construed in their inclusive, as opposed to their exclusive, sense, that is in the sense of “including, but not limited to”.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “symptom(s)” as used herein, refers to signs or indications that a subject is suffering from a specific condition or disease. For example, symptoms associated with SARS-COV-2 infection, as used herein, refer to signs or indications that a subject is infected with SARS-COV-2.

As used herein, the term “complication(s)” refers to a pathological process or event occurring during a disease or condition that is not an essential part of the disease or condition; where it may result from the disease/condition or from independent causes.

As used herein, the term “effective amount” in the context of administering a therapy to a subject refers to the amount of a therapy which has a prophylactic and/or therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of SARS-COV-2 infection, disease or symptom associated therewith; (ii) reduce the duration of SARS-COV-2 infection, disease or symptom associated therewith; (iii) prevent the progression of SARS-COV-2 infection, disease or symptom associated therewith; (iv) cause regression of SARS-COV-2 infection, disease or symptom associated therewith; (v) prevent the development or onset of SARS-COV-2 infection, disease or symptom associated therewith; (vi) prevent the recurrence of SARS-COV-2 infection, disease or symptom associated therewith; (vii) reduce or prevent the spread of SARS-COV-2 from one cell to another cell, one tissue to another tissue, or one organ to another organ; (ix) prevent or reduce the spread of SARS-COV-2 from one subject to another subject; (x) reduce organ failure associated with SARS-COV-2 infection; (xi) reduce hospitalization of a subject; (xii) reduce hospitalization length; (xiii) increase the survival of a subject with SARS-COV-2 infection or disease associated therewith; (xiv) eliminate SARS-COV-2 infection or disease associated therewith; (xv) inhibit or reduce SARS-COV-2 replication; (xvi) inhibit or reduce the entry of SARS-COV-2 into a host cell(s); (xviii) inhibit or reduce replication of the SARS-CoV-2 genome; (xix) inhibit or reduce synthesis of SARS-COV-2 proteins; (xx) inhibit or reduce assembly of SARS-COV-2 particles; (xxi) inhibit or reduce release of SARS-COV-2 particles from a host cell(s); (xxii) reduce SARS-COV-2 titre or viral load; and/or (xxiii) enhance or improve the prophylactic or therapeutic effect(s) of another therapy.

In certain embodiments, the effective amount does not result in complete protection from SARS-COV-2 infection, but results in a lower titre or viral load, reduced number of SARS-COV-2 or a lower viral load compared to an untreated subject. In certain embodiments, the effective amount results in a 0.5 fold, 1 fold, 2 fold, 4 fold, 6 fold, 8 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titre or viral load of SARS-COV-2 relative to an untreated subject. In some embodiments, the effective amount results in a reduction in titre or viral load of SARS-COV-2 compared to an untreated subject of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titre, viral load, number or total burden of SARS-COV-2 infection include, but are not limited to, less severe symptoms of the infection, fewer symptoms of the infection and a reduction in the length of the disease associated with the infection.

“Concurrent administration”, “concurrently administering”, “co-administration”, “co-administered” and the like as used herein, includes administering BIT225, or a pharmaceutically acceptable salt thereof, and one or more additional viral therapeutics together in a manner suitable for the treatment of SARS-COV-2 infection or for the treatment of SARS-COV-2 infection-related symptoms/complications. As contemplated herein, concurrent administration includes providing to a subject BIT225, or a pharmaceutically acceptable salt thereof, and one or more additional viral therapeutics as separate compounds, such as, for example, separate pharmaceutical compositions administered consecutively, simultaneously, or at different times. Preferably, if BIT225, or a pharmaceutically acceptable salt thereof, and one or more additional viral therapeutic are administered separately, they are not administered so distant in time from each other that BIT225, or a pharmaceutically acceptable salt thereof, and the one or more additional viral therapeutic cannot interact. BIT225, or a pharmaceutically acceptable salt thereof, and one or more additional viral therapeutic may be administered in any order. In one embodiment, BIT225, or a pharmaceutically acceptable salt thereof, can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration the one or more additional viral therapeutics to a subject. According to the subject invention, concurrent administration also encompasses providing one or more additional viral therapeutics in admixture with BIT225, or a pharmaceutically acceptable salt thereof, such as in a pharmaceutical composition.

An additional viral therapeutic of the invention includes vaccinations or antiviral medications such as a neuraminidase or hemagglutinin inhibitor or medications that modulate the immune system or host cell factors. Contemplated viral therapeutics for use in accordance with the subject invention include, but are not limited to, amantadine, rimantadine, ribavirin, idoxuridine, trifluridine, vidarabine, acyclovir, ganciclovir, foscarnet, zidovudine, didanosine, peramivir, zalcitabine, stavudine, famciclovir, oseltamivir, zanamivir, and valaciclovir.

In related embodiments, a subject diagnosed with SARS-COV-2 infection, BIT225, or a pharmaceutically acceptable salt thereof, may be concurrently administered with other therapeutics useful in the treatment of symptoms associated with SARS-COV-2 infection. For example, antitussives, mucolytics, expectorants, antipyretics, analgesics, or nasal decongestants can be concurrently administered with BIT225, or a pharmaceutically acceptable salt thereof, to a subject diagnosed with SARS-COV-2 infection.

As used herein, the term “infection” means the invasion by, multiplication and/or presence of a virus in a cell or a subject. In one embodiment, an infection is an “active” infection, i.e., one in which the virus is replicating in a cell or a subject. Such an infection is characterized by the spread of the virus to other cells, tissues, and/or organs, from the cells, tissues, and/or organs initially infected by the virus. An infection may also be a latent infection, i.e., one in which the virus is dormant.

As used herein, the expression “treating SARS-COV-2 infection” means improving, reducing, or alleviating at least one symptom or biological consequence of SARS-COV-2 infection in a subject, and/or reducing or decreasing SARS-COV-2 titer, load, replication or proliferation in a subject following exposure to SARS-COV-2. The expression “treating SARS-CoV-2 infection” also includes shortening the time period during which a subject exhibits at least one symptom or biological consequence of SARS-COV-2 infection. Methods for treating SARS-COV-2 infection, according to the present invention, comprise administering a pharmaceutical composition of the present invention to a subject after the subject is infected with SARS-COV-2 and/or after the subject exhibits or is diagnosed with one or more symptoms or biological consequences of SARS-COV-2 infection.

As used herein, the expression “preventing SARS-COV-2 infection” means preventing at least one symptom or biological consequence of SARS-COV-2 infection in a subject, and/or inhibiting or attenuating the extent to which SARS-COV-2 is capable of entering, spreading, and/or propagating within/among cells of an animal body. The expression “preventing SARS-COV-2 infection” also includes decreasing the susceptibility of a subject to at least one symptom or biological consequence of SARS-COV-2 infection. Methods for preventing SARS-COV-2 infection (i.e., prophylaxis) comprise administering a pharmaceutical composition of the present invention to a subject before the subject is infected with SARS-COV-2 and/or before the subject exhibits one or more symptoms or biological consequences of SARS-COV-2 infection. Methods for preventing SARS-COV-2 infection may include administering a pharmaceutical composition of the present invention to a subject at a particular time period or season of the year (e.g., during the 1-2 month period just prior to the time at which peak numbers of individuals are typically found to experience SARS-COV-2 infection), or before the subject travels to or is exposed to an environment with high frequencies of SARS-COV-2 infection, and/or before the subject is exposed to other subjects who are infected with SARS-COV-2.

As used herein, the terms “replication,” “viral replication” and “virus replication” in the context of a virus refer to one or more, or all, of the stages of a viral life cycle which result in the propagation of virus. The steps of a viral life cycle include, but are not limited to, virus attachment to the host cell surface, penetration or entry of the host cell (e.g., through receptor mediated endocytosis or membrane fusion), uncoating (the process whereby the viral capsid is removed and degraded by viral enzymes or host enzymes thus releasing the viral genomic nucleic acid), genome replication, synthesis of viral messenger RNA (mRNA), viral protein synthesis, and assembly of viral ribonucleoprotein complexes for genome replication, assembly of virus particles, post-translational modification of the viral proteins, and release from the host cell by lysis or budding and acquisition of a phospholipid envelope which contains embedded viral glycoproteins. In some embodiments, the terms “replication,” “viral replication” and “virus replication” refer to the replication of the viral genome. In other embodiments, the terms “replication,” “viral replication” and “virus replication” refer to the synthesis of viral proteins.

As used herein, the term “titre” in the context of a virus refers to the number of viral particles present in a given volume of blood or other biological fluid or tissue or organ weight. The terms “viral load” and “viral burden” may also be used.

As used herein, the term “COVID-19” refers to the disease caused by SARS-COV-2.

As used herein, the term “subject” is used to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In some embodiments, a subject is a farm animal or pet. In another embodiment, a subject is a human. In another embodiment, a subject is a human infant. In another embodiment, a subject is a human child. In another embodiment, a subject is a human adult. In another embodiment, a subject is an elderly human. In another embodiment, a subject is a premature human infant.

The pharmaceutical compositions of the invention may be in the form of a liposome or micelles in which compounds of the present invention are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

Routes of administration include, but are not limited to, intravenous, intraperitoneal, subcutaneous, intracranial, intradermal, intramuscular, intraocular, intrathecal, intracerebral, intranasal, transmucosal, or by infusion orally, rectally, via iv drip, patch and implant. Oral routes are particularly preferred.

Compositions suitable for injectable use include sterile aqueous solutions (where water soluble) and sterile powders for the extemporaneous preparation of sterile injectable solutions. The carrier can be a solvent or dispersion medium containing, for example, water, ethanol, polyol (for example, glycerol, propylene glycol and liquid polyethylene glycol, and the like), suitable mixtures thereof and vegetable oils. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by, for example, filter sterilization or sterilization by other appropriate means. Dispersions are also contemplated and these may be prepared by incorporating the various sterilized active ingredients into a sterile vehicle which contains the basic dispersion medium and the required other ingredients from those enumerated above. In the case of sterile powders for the preparation of sterile injectable solutions, a preferred method of preparation includes vacuum drying and the freeze-drying technique which yield a powder of the active ingredient plus any additional desired ingredient from a previously sterile-filtered solution.

When the active ingredients are suitably protected, they may be orally administered, for example, with an inert diluent or with an assimilable edible carrier, or it may be enclosed in hard or soft shell gelatin capsule, or it may be compressed into tablets. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 0.01% by weight, more preferably 0.1% by weight, even more preferably 1% by weight of active compound. The percentage of the compositions and preparations may, of course, be varied and may conveniently be between about 1 to about 99%, more preferably about 2 to about 90%, even more preferably about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 ng and 2000 mg of active compound.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Any material used in preparing any dosage unit form should be pharmaceutically pure and substantially non-toxic in the amounts employed. In addition, the active compound(s) may be incorporated into sustained-release preparations and formulations.

The present invention also extends to forms suitable for topical application such as creams, lotions and gels. In such forms, components may be added or modified to assist in penetration of the surface barrier.

Procedures for the preparation of dosage unit forms and topical preparations are readily available to those skilled in the art from texts such as Pharmaceutical Handbook, 19th edition (Edited by Ainley Wade), The Pharmaceutical Press London; CRC Handbook of Chemistry and Physics (edited by Robert C. Weast), CRC Press Inc.; Goodman and Gilman's The Pharmacological basis of Therapeutics, 9th edition, McGraw Hill; Remington: The Science and Practice of Pharmacy, 19th edition (edited by Joseph P. Remington and Alfonso R. Gennaro), Mack Publishing Co.

The term “pharmaceutically acceptable salt,” as used herein, refers to any salt of BIT225 that is pharmaceutically acceptable and does not greatly reduce or inhibit the activity BIT225. Suitable examples include acid addition salts, with an organic or inorganic acid such as acetate, tartrate, trifluoroacetate, lactate, maleate, fumarate, citrate, methane, sulfonate, sulfate, phosphate, nitrate, or chloride.

Pharmaceutically acceptable carriers and/or diluents include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved and (b) the limitations inherent in the art of compounding.

Effective amounts contemplated by the present invention will vary depending on the severity of the condition and the health and age of the recipient. In general terms, effective amounts may vary from 0.01 ng/kg body weight to about 100 mg/kg body weight. Effective amounts include about 100 mg to about 600 mg, in particular about 100 mg, about 150 mg, about 200 mg, about 250 mg, about 300 mg, about 350 mg, about 400 mg, about 450 mg, about 500 mg, about 550 mg, or about 600 mg.

Other than in the operating examples, or where otherwise indicated, all numbers expressing quantities of ingredients or reaction conditions used herein are to be understood as modified in all instances by the term “about”.

As used herein the term “about” can mean within 1 or more standard deviation per the practice in the art. Alternatively, “about” can mean a range of up to 20%. When particular values are provided in the specification and claims the meaning of “about” should be assumed to be within an acceptable error range for that particular value.

The recitation of a numerical range using endpoints includes all numbers subsumed within that range (e.g., 1 to 5 includes 1, 1.5, 2, 2.75, 3, 3.80, 4, 5, etc.).

### Preferred Embodiment of the Invention

Although the invention has been described with reference to certain embodiments detailed herein, other embodiments can achieve the same or similar results. Variations and modifications of the invention will be obvious to those skilled in the art and the invention is intended to cover all such modifications and equivalents.

The present application is based on the surprising finding that BIT225 has activity against SARS-COV-2.

The present invention provides methods and compositions (such as pharmaceutical compositions) for treating or preventing SARS-COV-2 infection or resultant COVID-19.

The present invention provides materials and methods for preventing and/or treating viral infections. Specifically, the subject invention provides materials and methods for preventing SARS-COV-2 infection; treating/ameliorating symptoms associated with SARS-CoV-2 infections; and/or preventing/delaying the onset of complications associated with SARS-COV-2 infections.

The present invention provides a method for the treatment or prevention of SARS-CoV-2 infection in a subject, the method comprising administering to the subject an effective amount of BIT225, or a pharmaceutically acceptable salt thereof.

The present invention also provides a method for inhibiting the replication of SARS-CoV-2 in a subject, the method comprising administering to the subject an effective amount BIT225, or a pharmaceutically acceptable salt thereof.

The present invention further provides a method for reducing the severity, intensity, or duration of complications or symptoms associated with SARS-COV-2 infection in a subject, the method comprising administering to the subject an effective amount BIT225, or a pharmaceutically acceptable salt thereof.

The invention further provides a method of reducing the viral load of SARS-COV-2 in a subject, the method comprising administering to the subject an effective amount of BIT225, or a pharmaceutically acceptable salt thereof.

The invention further provides a method of reducing the production of a proinflammatory cytokine or chemokine in a subject infected with SARS-COV-2, the method comprising administering to the subject an effective amount of BIT225, or a pharmaceutically acceptable salt thereof.

The present invention is further described by the following non-limiting examples.

### EXAMPLES

### Example 1—Production of BIT225

A mixture of 5-bromo-2-naphthoic acid (2.12 g, 8.44 mmol), 1-methyl-4-(4,4,5,5-tetramethyl-1,3,2-dioxaborolan-2-yl)-1H-pyrazole (1.84 g, 8.86 mmol), and tetrakis (triphenylphosphine) palladium (0) (502 mg, 0.435 mmol) in a 250 mL round bottomed flask was evacuated and purged with nitrogen (in three cycles). Acetonitrile (40 mL) and 2M aqueous sodium carbonate (10 mL) were added to the mixture via syringe, and the mixture was heated under reflux under nitrogen for 22 hours. The reaction mixture was allowed to cool before the addition of 1M aqueous hydrochloric acid (30 mL) and it was then extracted with ethyl acetate (3×50 mL). The combined organic layers were dried (MgSO₄), filtered, and concentrated in vacuo to provide a crude product (2.98 g after air drying). This crude material was dissolved in hot ethanol (150 mL) and filtered while hot to remove a yellow impurity (120 mg). The filtrate was concentrated in vacuo and the residue was recrystallised from dichloromethane (30 mL) to provide 5-(1-methyl-1H-pyrazol-4-yl)-2-naphthoic acid as a white solid (724 mg, 34%). A second crop of 5-(1-methyl-1H-pyrazol-4-yl)-2-naphthoic acid (527 mg, 25%) was obtained from the concentrated mother liquors by recrystallisation from dichloromethane (20 mL).

Oxalyl chloride (1.1 mL, 13 mmol) was added to the solution of 5-(1-methyl-1H-pyrazol-4-yl)-2-naphthoic acid (1.19 g, 4.71 mmol) in anhydrous dichloromethane (200 mL (which was added in portions during the reaction to effect dissolution)) containing dimethylformamide (2 drops) under nitrogen and the mixture was stirred at room temperature for 4.25 hours. The reaction mixture was then heated for 1 hour at 40° C., before being concentrated under reduced pressure. The resulting crude acid chloride was suspended in anhydrous tetrahydrofuran (50 mL) and this mixture was added dropwise to a solution of guanidine hydrochloride (2.09 g, 21.9 mmol) in 2M aqueous sodium hydroxide (15 mL, 30 mmol) and the reaction mixture was then stirred for 30 minutes. The organic phase was separated, and the aqueous phase was extracted with chloroform (3×30 mL) followed by ethyl acetate (3×30 mL). The combined organic extracts were washed sequentially with 1M aqueous sodium hydroxide (60 mL) and water (40 mL), then dried (Na₂SO₄) and concentrated in vacuo to give a glassy solid (1.45 g after drying under high vacuum). This solid was dissolved in dichloromethane which was then allowed to evaporate slowly to give BIT225 as a yellow solid (1.15 g, 83%).

### Example 2—In Vitro Assays

Virus: “US” US-WA1/2020 (NR-52281),
   “Delta” US-PHC658/2021 (delta variant; NR-55611),
   “SA” SouthAfrica/KRISP-K005325/2020 (beta variant; NR-54009),
   “UK” England/204820464/2020 (alpha variant; NR-54000),
   “BR” Japan/TY7-503/2021-Brazil_P.1 (NR-54982) and
   “Omicron” USA/MD-HP20874/2021 (omicron variant; NR-56461)
Infected cells: Vero (African Green Monkey kidney) and Calu 3 (human lung epithelial cancer)
BIT225 concentrations: 0, 0.3, 0.6, 1.25, 2.5, 5 & 10 UM (in triplicate)
BIT225 added 1 hour pre-infection

Strains were obtained from BEI Resources (Manassas, VA, USA). Virus was passaged in Vero E6 cells or Calu 3 cells, maintained at 37° C. and 5% CO₂ in Dulbecco's Modified Eagles Medium supplemented with 10% foetal bovine serum.

The number of viral genome copies released by Vero and Calu 3 cells into culture medium at 4 days post infection was measured by qRT-PCR (copies/ml (log 10)) using the method described by Winkler et al. 2020 (Nat. Immunol. 21:1327-1335). Briefly, total viral RNA was extracted from test culture medium using the MagMax™ mirVana™ Total RNA Isolation Kit (ThermoFisher Scientific, Waltham, MA, USA) on the KingFisher™ Flex extraction robot (ThermoFisher Scientific, Waltham, MA, USA). The SARS-COV-2 nucleocapsid (N) gene was reverse transcribed and amplified using the TaqMan® RNA-to CT™ 1-Step Kit (ThermoFisher Scientific, Waltham, MA, USA). The SARS-COV-2 N gene was detected using:

Forward primer:
ATGCTGCAATCGTGCTACAA;
Reverse primer:
GACTGCCGCCTCTGCTC;
Probe:
56FAM/TCAAGGAAC/ZEN/AACATTGCCAA/3IABKFQ.

The amount of infectious virus released by Vero and Calu 3 cells into culture medium at 4 days post infection was determined by plaque assay (PFU/ml (log 10)) using the method described in van den Worm et al. 2012 (PLOS One 7 (3): e32857). Briefly, Vero-E6 cells in 6-well clusters were incubated at 37° C. for 1 hour with test culture medium diluted in PBS containing DEAE (0.005% w/v) and 2% FCS. Subsequently, the test culture medium was replaced with 2 ml of a 1.2% suspension of Avicel (RC-581; FMC Biopolymer) in DMEM containing 2% FBS, 25 mM HEPES, penicillin (100 IU/ml) and streptomycin (100 IU/ml). Cells were incubated at 37° C. for 48-60 hours and fixed with formaldehyde, after which plaques were visualised using crystal violet staining.

For dose-response curves, R package (drc) was used to generate the plots and for estimation of EC50 values. All plotting and statistical analysis was performed using R statistical software, version 4.0.4 (R_Core_Team. 2021. R: A language and environment for statistical computing. R Foundation for Statistical Computing, URL https://www.R-project.org/).

Dose response curves were fit and analysed using R package, drc (Ritz C, Baty F, Streibig JC, Gerhard D. 2015. Dose-Response Analysis Using R. PLOS One 10: e0146021). Three-parameter log-logistic models (equation 1) were fit to the concentration (x) versus response (f(x)) data via function drm( . . . , fct=LL.3) and the “delta” method and t-distribution were used to estimate EC50 values (parameter e in Eqn 1) and asymptotic-based confidence intervals.

$$f ( x , ( b , d , e ) ) = \frac{d}{1 + exp ( b ( log ( x ) - log ( e ) ) )} \qquad Eqn . 1$$

(where, x, f (x) and parameter e are as described above; d and b are parameters capturing the maximum and slope of the best-fit response curve, respectively).

Experiments were done in a dose-response format and virus released to culture medium was quantitated by qRT-PCR (for genome copies) and plaque assay (for infectious virus). Monolayers were exposed to test concentrations of BIT225 for 1 hour prior to infection with SARS-COV-2 at a multiplicity of infection (m.o.i) of 0.1.

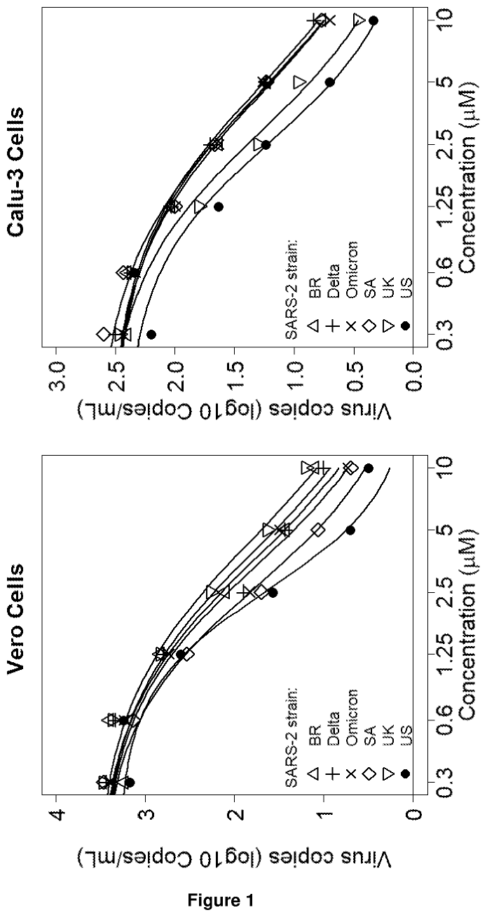

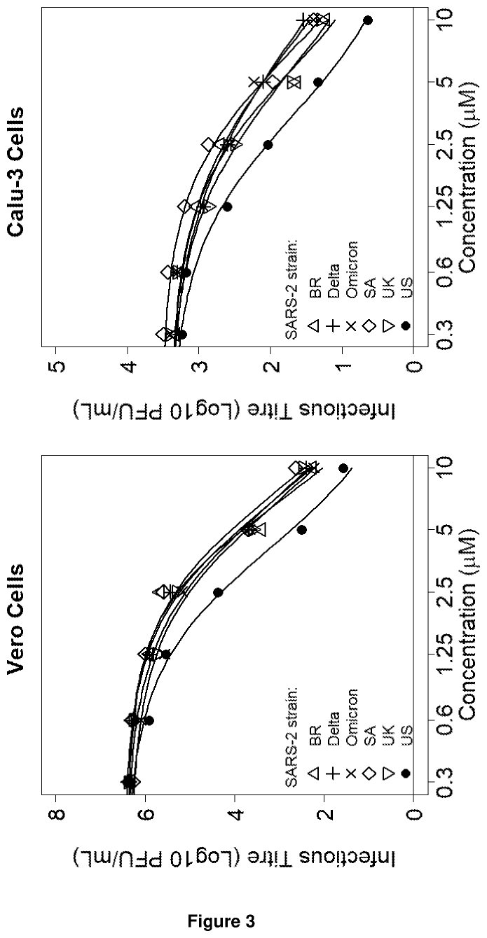

The results demonstrated that BIT225 inhibits production and release of SARS-CoV-2 from infected monolayer cultures of Vero-E6 cells and Calu-3 cells. Dose response curves (FIGS. 1 and 3) show that BIT225 has similar antiviral efficacy against all six strains of SARS-COV-2 tested and in both cell types. Table 1 lists the parameter estimates for BIT225 (EC50; maximum response; and Hill slope) derived from the log-logistic curve fits (parameters e, d & b, respectively, in Eqn. 1).

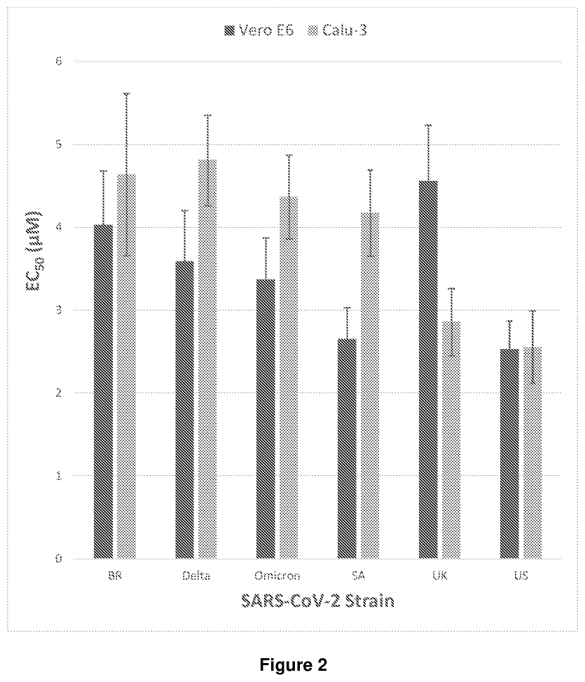

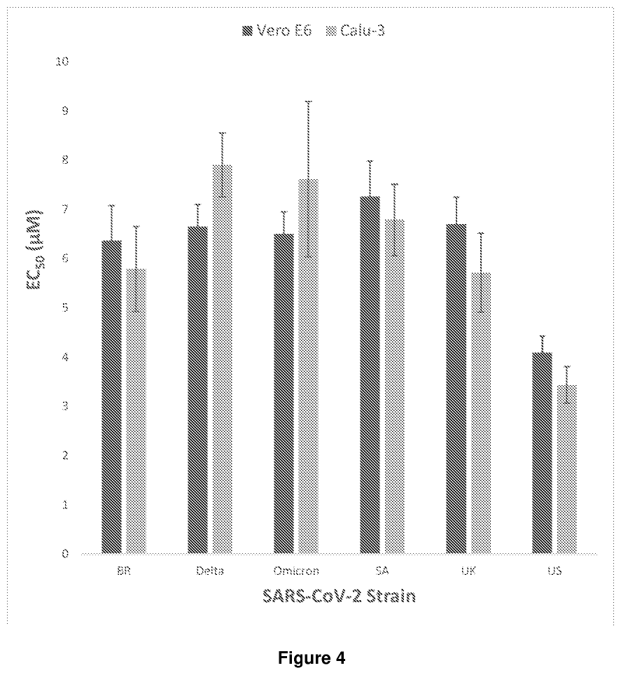

For the qRT-PCR assay (FIGS. 1 and 2), BIT225 response curves were similar for the six strains with EC50 estimates ranging between 2.5 μM and 4.8 UM across the two cell types (mean 3.7 μM). For the plaque assay (FIGS. 3 and 4), EC50 ranged between 3.4 UM and 7.9 μM (mean 6.2 μM) and two-way ANOVA found the EC50 for strain US (3.8 μM) was lower than estimates for the Delta, Omicron and Beta variants (P<0.05, Tukey adjusted). ANOVA of the maximum response and Hill slope estimates (Table 1) found that, while the mean values of both parameters are slightly lower in Calu-3 cells than Vero, these parameters are not significantly different between the virus strains.

TABLE 1
Effect of BIT225 on virus release and infectious virus produced

| | | | EC₅₀ | | Max. Response (d) | | Slope (b) | |
|---|---|---|---|---|---|---|---|---|
| Assay | Cell | Virus | (μM) | [C.I. 95%] | (log10) | [C.I. 95%] | (nmol⁻¹) | [C.I. 95%] |
| PCR | Calu-3 | WA1 | 2.55 | [2.12, 2.99] | 2.41 | [2.28, 2.55] | 1.38 | [1.12, 1.64] |
| | | Delta | 4.81 | [4.26, 5.35] | 2.56 | [2.48, 2.64] | 1.08 | [0.95, 1.21] |
| | | Omicron | 4.37 | [3.86, 4.87] | 2.57 | [2.49, 2.65] | 1.08 | [0.95, 1.21] |
| | | BR | 4.64 | [3.66, 5.61] | 2.53 | [2.38, 2.68] | 1.12 | [0.86, 1.38] |
| | | SA | 4.17 | [3.65, 4.69] | 2.67 | [2.58, 2.76] | 1.07 | [0.93, 1.20] |
| | | UK | 2.86 | [2.45, 3.26] | 2.58 | [2.47, 2.69] | 1.21 | [1.04, 1.37] |
| | Vero | WA1 | 2.53 | [2.19, 2.88] | 3.29 | [3.13, 3.45] | 1.79 | [1.42, 2.16] |
| | | Delta | 3.59 | [2.98, 4.21] | 3.55 | [3.39, 3.72] | 1.15 | [0.96, 1.35] |
| | | Omicron | 3.37 | [2.87, 3.87] | 3.51 | [3.36, 3.66] | 1.22 | [1.04, 1.40] |
| | | BR | 4.03 | [3.38, 4.68] | 3.53 | [3.37, 3.68] | 1.13 | [0.94, 1.32] |
| | | SA | 2.65 | [2.27, 3.03] | 3.52 | [3.36, 3.67] | 1.28 | [1.10, 1.47] |
| | | UK | 4.56 | [3.89, 5.24] | 3.58 | [3.44, 3.72] | 1.07 | [0.91, 1.23] |
| Plaque | Calu-3 | WA1 | 3.43 | [3.06, 3.81] | 3.38 | [3.27, 3.50] | 1.31 | [1.15, 1.48] |
| | | Delta | 7.9 | [7.26, 8.55] | 3.44 | [3.38, 3.51] | 1.00 | [0.90, 1.09] |
| | | Omicron | 7.61 | [6.04, 9.19] | 3.42 | [3.24, 3.60] | 1.11 | [0.81, 1.41] |
| | | BR | 5.79 | [4.93, 6.65] | 3.39 | [3.25, 3.53] | 1.36 | [1.08, 1.64] |
| | | SA | 6.79 | [6.06, 7.51] | 3.52 | [3.42, 3.62] | 1.34 | [1.14, 1.55] |
| | | UK | 5.72 | [4.91, 6.52] | 3.43 | [3.30, 3.55] | 1.15 | [0.96, 1.33] |
| | Vero | WA1 | 4.09 | [3.75, 4.44] | 6.40 | [6.23, 6.56] | 1.45 | [1.29, 1.62] |
| | | Delta | 6.65 | [6.2, 7.11] | 6.45 | [6.33, 6.58] | 1.48 | [1.32, 1.64] |
| | | Omicron | 6.5 | [6.05, 6.95] | 6.31 | [6.18, 6.43] | 1.41 | [1.26, 1.57] |
| | | BR | 6.36 | [5.64, 7.07] | 6.39 | [6.17, 6.60] | 1.67 | [1.36, 1.98] |
| | | SA | 7.26 | [6.54, 7.97] | 6.40 | [6.23, 6.57] | 1.50 | [1.26, 1.73] |
| | | UK | 6.7 | [6.15, 7.26] | 6.41 | [6.26, 6.55] | 1.38 | [1.21, 1.55] |

### Example 3—Mouse Studies

Six-to-eight-week-old transgenic mice expressing human ACE2 under the control of the cytokeratin 18 promoter (K18-hACE2 mice) were purchased from The Jackson Laboratory (Bar Harbor, ME, USA; Stock No. 034860) and assessed for ill health upon arrival. The animals underwent acclimatisation for 1-2 weeks and were housed individually to minimize the risk of cross infection. Animals were maintained under isoflurane anaesthesia for dosing and virus inoculation and were returned to their housing for recovery.

The 2019n-COV/US-WA1/2020 strain of SARS-COV-2 was used in these studies (obtained from BEI Resources (Manassas, VA, USA), National Institute of Allergy and Infectious Disease (NIAID)). The virus was passaged in Vero E6 cells (CRL-1586™, ATCC, Washington, DC, USA). The Vero E6 cells were maintained in Dulbecco's Modified Eagle's Medium (DMEM) supplemented with 10% foetal bovine serum (FBS).

The quantitation of viral inoculum generated in vitro and the amount of virus in tissue homogenates at the end of the study were determined by plaque assay in Vero E6 cells. Viral genomes in serum and tissue homogenates were detected by a quantitative reverse transcriptase polymerase chain reaction (qRT-PCR). Virus strain: 2019n-COV/USA-WA1/2020

The mice were inoculated via intranasal administration with 104 PFU of SARS-CoV-2 (2019n-COV/USA-WA1/2020) on Day 1 and treated by oral gavage as follows:
Experiment 1, Group 1:5 mice inoculated via intranasal administration with 104 PFU of SARS-COV-2 (2019n-COV/USA-WA1/2020) on Day 1 and treated by oral gavage with 100 mg/kg BIT225 (dose volume of 3 ml/kg; 33.4 mg/ml in vehicle twice daily for 7 days.
Experiment 1, Group 2:5 mice inoculated via intranasal administration with 104 PFU of SARS-COV-2 (2019n-COV/USA-WA1/2020) on Day 1 and treated by oral gavage with 300 mg/kg BIT225 (dose volume of 3 ml/kg; 100 mg/ml in vehicle) twice daily for 7 days.
Experiment 1, Group 3:5 mice inoculated via intranasal administration with 104 PFU of SARS-COV-2 (2019n-COV/USA-WA1/2020) on Day 1 and treated by oral gavage with vehicle control (dose volume of 3 ml/kg; vehicle) twice daily for 7 days.
Experiment 2, Group 1:7 mice inoculated via intranasal administration with 104 PFU of SARS-COV-2 (2019n-COV/USA-WA1/2020) on Day 1 and treated by oral gavage with 300 mg/kg BIT225 twice daily for 12 days.
Experiment 2, Group 2:7 mice inoculated via intranasal administration with 104 PFU of SARS-COV-2 (2019n-COV/USA-WA1/2020) on Day 1 and treated by oral gavage with vehicle control twice daily for 12 days.
Experiment 3, Group 1:4 mice inoculated via intranasal administration with 104 PFU of SARS-COV-2 (2019n-COV/USA-WA1/2020) on Day 1 and treated by oral gavage with 300 mg/kg BIT225 twice daily for 5 days.
Experiment 3, Group 2:4 mice inoculated via intranasal administration with 104 PFU of SARS-COV-2 (2019n-COV/USA-WA1/2020) on Day 1 and treated by oral gavage with vehicle control twice daily for 5 days.
Experiment 4, Group 1:5 mice treated with 300 mg/kg BIT225 twice daily commencing 24 hours before inoculation via intranasal administration with 104 PFU of SARS-COV-2 (2019n-COV/USA-WA1/2020).
Experiment 4, Group 2:5 mice treated with 300 mg/kg BIT225 twice daily commencing 24 hours after inoculation via intranasal administration with 104 PFU of SARS-COV-2 (2019n-CoV/USA-WA1/2020).
Experiment 4, Group 3:5 mice treated with 300 mg/kg BIT225 twice daily commencing 48 hours before inoculation via intranasal administration with 104 PFU of SARS-COV-2 (2019n-COV/USA-WA1/2020).
Experiment 4, Group 4:5 mice treated with vehicle control twice daily commencing 24 hours before inoculation via intranasal administration with 104 PFU of SARS-CoV-2 (2019n-COV/USA-WA1/2020).

The vehicle control was:
0.5% (w/v) hydroxypropyl methylcellulose,
0.5% (v/v) benzyl alcohol and
0.4% (v/v) polysorbate 80 in Milli-Q water (HPMC-SV).

Body weights were recorded prior to the first dose each morning. For mortality, loss of >30% body weight compared to Day 1 pre-inoculation weight was pre-determined (under ethical considerations) as a trigger for immediate euthanasia. Mice that survived to the planned termination times (Day 5, Day 7, or Day 12 in different experiments) were euthanised and lungs and blood samples were harvested for quantitation of virus genome copy number, infectious virus titre and cytokine concentrations.

Percent weight change from Day 1 (pre-infection) were calculated for each mouse at each time point (equation 2).

$$Weight {change}_{Day . i} ( \% ) = \frac{{weight}_{Day . i} - {weight}_{Day \text{.1}}}{{weight}_{Day \text{.1}}} * 100 \% \qquad Eqn . 2$$

Group mean at time points were compared by two-sided Welch's T-tests. Between group comparisons of weight change, virus genome copy number, infectious titre and cytokine levels also used two-sided Welch's T-tests.

For mortality comparisons, standard Kaplan-Meier analysis (with right-censoring) was performed using R's package: survival (v3.2-13; Therneau, T. M. (2020). A Package for Survival Analysis in R. https://CRAN.R-project.org/package=survival) and survival curves were compared by the log-rank test.

The number of viral genome copies in blood and lung tissue homogenate was determined by qRT-PCR using the method described by Winkler et al. 2020 (Nat. Immunol. 21:1327-1335). Briefly, total viral RNA was extracted from serum or tissue using the MagMax™ mirVana™ Total RNA Isolation Kit (ThermoFisher Scientific, Waltham, MA, USA) on the KingFisher™ Flex extraction robot (ThermoFisher Scientific, Waltham, MA, USA). The SARS-COV-2 nucleocapsid (N) gene was reverse transcribed and amplified using the TaqMan® RNA- to CT™ 1-Step Kit (ThermoFisher Scientific, Waltham, MA, USA). The SARS-COV-2 N gene was detected using:

Forward primer:
ATGCTGCAATCGTGCTACAA;
Reverse primer:
GACTGCCGCCTCTGCTC;
Probe:
56FAM/TCAAGGAAC/ZEN/AACATTGCCAA/3IABKFQ.

The amount of infectious virus in blood and lung tissue homogenate was determined by plaque assay using the method described in van den Worm et al. 2012 (PLOS One 7 (3): e32857). Briefly, Vero-E6 cells in 6-well clusters were incubated at 37° C. for 1 hour with blood or lung homogenate in PBS containing DEAE (0.005% w/v) and 2% FCS. Subsequently, the blood and lung tissue homogenate were replaced with 2 ml of a 1.2% suspension of Avicel (RC-581; FMC Biopolymer) in DMEM containing 2% FBS, 25 mM HEPES, penicillin (100 IU/ml) and streptomycin (100 IU/ml). Cells were incubated at 37° C. for 48-60 hours and fixed with formaldehyde, after which plaques were visualised using crystal violet staining.

Disease markers measured were survival; percent weight change from pre-infection baseline; viral loads (qPCR assay); and infectious virus titre in lung tissue and serum samples (plaque assay). In addition, levels of six pro-inflammatory cytokines/chemokines (IL-6; IL-1a; IL-1B; TNFα; TGFβ; MCP-1) were measured in lung and serum samples.

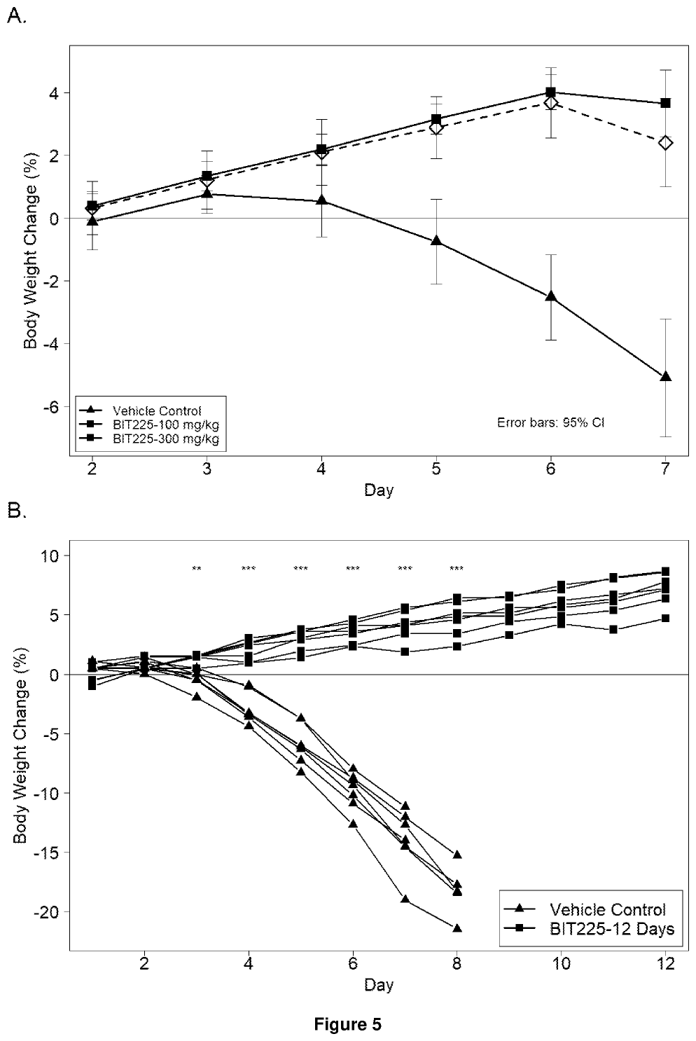

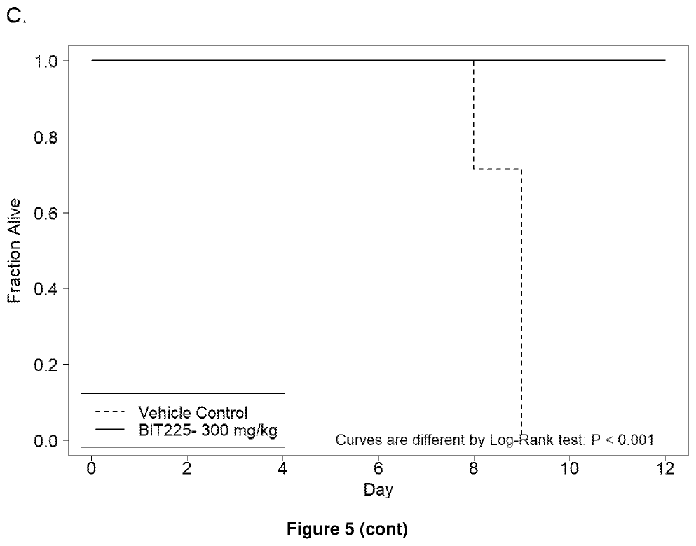

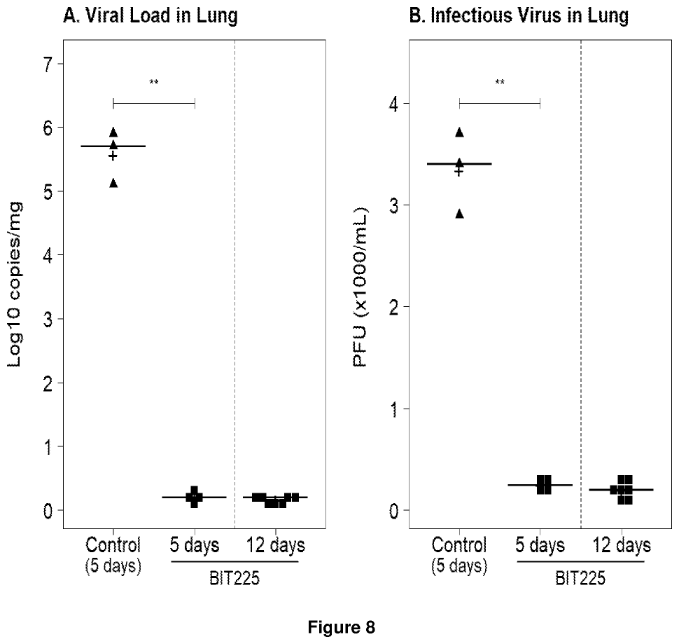

Three experiments with different dosing regimen and durations were performed to compare disease endpoints in BIT225 and vehicle dosing groups. Experiment 1 compared vehicle with two levels of BIT225 (100 mg/kg and 300 mg/kg) over seven days of dosing in groups of 5 mice (FIG. 5A). Experiment 2 was extended to 12 days dosing to include mortality analysis and compare vehicle with BIT225 (300 mg/kg) over 12 days of dosing in groups of 7 mice (FIGS. 5B & C). Experiment 3 involved sacrificing BIT225-dosed or vehicle control mice (n=4 per group) at Day 5 post-infection. Based on previous results, Day 5 was expected to be prior to death of any animals in the vehicle control group, however, one of the mice in the vehicle group died by Day 3. Nevertheless, three vehicle-group mice survived to Day 5 to enable comparison of viral load (FIG. 8) and cytokine/chemokine (data not shown) responses between the groups.

Across the three experiments, all 16 mice dosed with vehicle-only began losing body weight from day 3 post infection. In contrast, 21 out of 21 mice continued to gain body weight while receiving 12 hourly doses of BIT225. In Experiment 1 (FIG. 5A), there was no significant difference in body weight changes between the two BIT225 dosing levels (100 mg/kg or 300 mg/kg), and at day 7 all 10 BIT225-dosed mice had body weights greater than their pre-infection weights. The vehicle control group showed significant weight loss compared to either of the BIT225 groups (P<0.001; one-way ANOVA). On days 5, 6 & 7, the differences between group-mean body weight changes were statistically lower in the vehicle group compared to combined BIT225 groups: by 3.8% (P=0.01); 6.4% (P=0.001); and 8.1% (95% CI [5.6-10.7], P=0.001), respectively, (P values are from Welch's T-test, adjusted for tests at six time points).

In Experiment 2 (FIG. 5B), all mice in the vehicle control group (n=7) began to lose weight from day 3 post-infection. Two deaths occurred by the morning of day 8, and the remaining 5 mice in the control group died by day 9 (FIG. 5C). In contrast, for the group dosed with BIT225 for 12 days (n=7), all mice survived and continued to gain weight until euthanised on day 12 for tissue harvest. ANOVA and T-tests at individual days established strong statistical significance for the body weight differences between these two groups, starting from day 3 (adjusted P values as indicated in FIG. 5B). Experiment 3 (5 days dosing; n=4 each in BIT225 and control groups) gave the same trends for weight change as Experiment 1 and Experiment 2, with all mice receiving BIT225 remained healthy and gained weight to Day 5 (data not shown). In the control group, one mouse died by day 3 and the other three lost between 5% and 15% body weight by day 5. The Kaplan-Meier mortality curves for Experiment 2 are shown in FIG. 5C. The control and BIT225 curves are significantly different (P<0.001 by the log-rank test). Dosing with BIT225 provided a clear survival advantage.

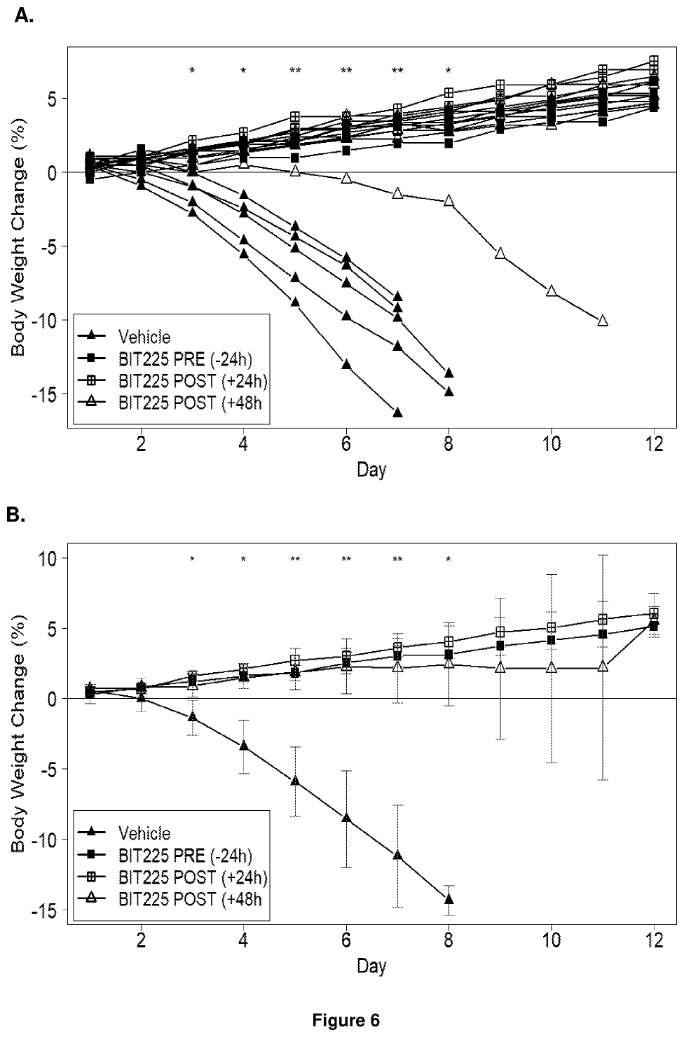

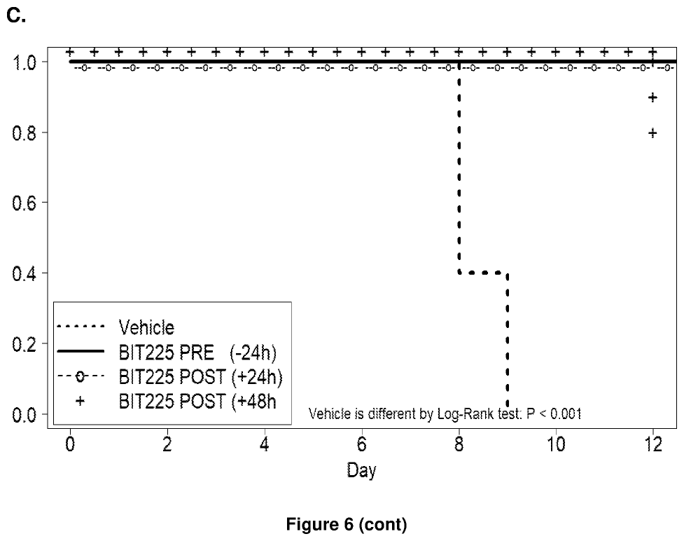

Experiment 4 compared the efficacy of initiating BIT225 dosing 24 hours pre-SARS-2-infection, 24 hours post-SARS-2-infection or 48 hours post-SARS-2-infection (FIG. 6). Four groups of 5 mice were infected with the same lethal inoculum (104 pfu) as the earlier experiments and dosed twice daily from 24 h pre-infection with either vehicle control or BIT225 (300 mg/kg). For the post-infection groups, dosing was switched from vehicle to BIT225 on the mornings of Day 2 (24 h.p.i) or day 3 (48 h.p.i). The Kaplan-Meier survival curves for this experiment are shown in FIG. 6 B: As in the previous studies, all mice in the vehicle control group died by day 8. All BIT225 pre-dosed mice (n=5) and 24-hour post-dosing mice (n=5) remained healthy and continued gain weight as per age expectations through to day 12. One of the five mice in the 48-hour post-dosing group began losing weight from Day 4 and died on day 11, while the other four mice in that cohort remained healthy and gained weight similarly to mice in the other BIT225 groups. From group-mean trendlines (not shown) there appears to be a trend for less weight gain as initiation of BIT225 dosing is delayed, but the difference between the pre-dosing and 48-hour post-dosing groups is not statistically significant at day 11 (P=0.3, T-test). Importantly, the 48-hour post-dosing group was clearly superior to the untreated group.

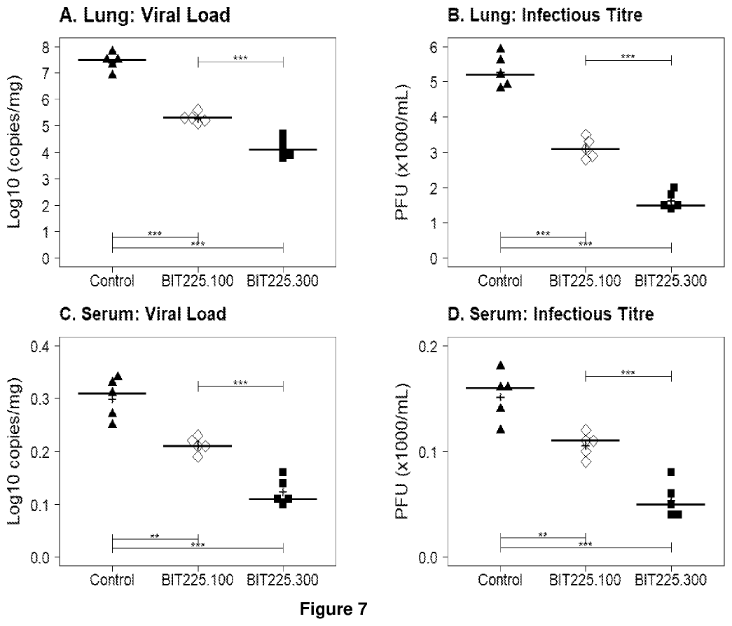

BIT225 was associated with significant reductions in both viral load and infectious virus in lung homogenates and serum in mice treated with BIT225 100 mg/kg or 300 mg/kg for 7 days (FIG. 7). In addition, virus reduction was dose responsive. In lung, 100 mg/kg dose gave approximately 2 log 10 reduction of viral load (P<0.001, T-test), while the 300 mg/kg dose gave approximately 3.5 log 10 reduction (P<0.001, between doses). Similarly, infectious virus recovered from lung tissue was reduced by approximately 2000 PFU/mL and 4000 PFU/mL, for the respective doses (P<0.001). These changes were also reflected in serum samples, albeit at lower absolute levels of virus detected.

Lung viral loads were also measured for mice that survived until day 5 (Experiment 3) or Day 12 (Experiment 2) (FIG. 8). The 11 mice treated with BIT225 (300 mg/kg twice daily) all survived and showed lung viral loads at or below the limits of detection for the qRT-PCR assay and very low plaque counts (<200/ml), while the three mice that survived to day 5 had viral loads of 105-106 copies/mg and generated approximately 3000 to 4000 plaques per mL of homogenate.

Inflammation was measured by determining amounts of the proinflammatory cytokines interleukin-6 (IL-6) (RayBio® Mouse IL-6 ELISA [ELM-IL6-1], RayBiotech Life, Peachtree Corners, GA, USA), interleukin-1alpha (IL-1α) (RayBio® Mouse IL-1 ELISA [ELM-IL1alpha-1]), interleukin-1beta (IL-1 β) (RayBio® Mouse IL-1 ELISA [ELM-IL1beta-1], RayBiotech Life, Peachtree Corners, GA, USA), tumour necrosis factor alpha (TNF-α) (RayBio® Mouse TNF-alpha ELISA [ELM-TNFα-1], RayBiotech Life, Peachtree Corners, GA, USA), transforming growth factor beta (TGF-β) (TGF-beta-1 Mouse ELISA kit [BMS6084], ThermoFisher Scientific, Waltham, MA, USA), and the proinflammatory chemokine monocyte chemoattractant protein-1 (MCP-1) (RayBio® Mouse MCP-1 ELISA [ELM-MCP1-1], RayBiotech Life, Peachtree Corners, GA, USA) according to their respective manufacturers' instructions. The ELISA protocols utilised a solid-phase sandwich ELISA design. A cytokine/chemokine target antibody had been precoated to the plate. The samples were added to the wells to bind to the capture antibody. The addition of a second antibody enabled the detection of the target-antibody sandwich complex, which was quantitated using a colorimetric reporting signal that was directly proportional to the concentration in the original specimen.

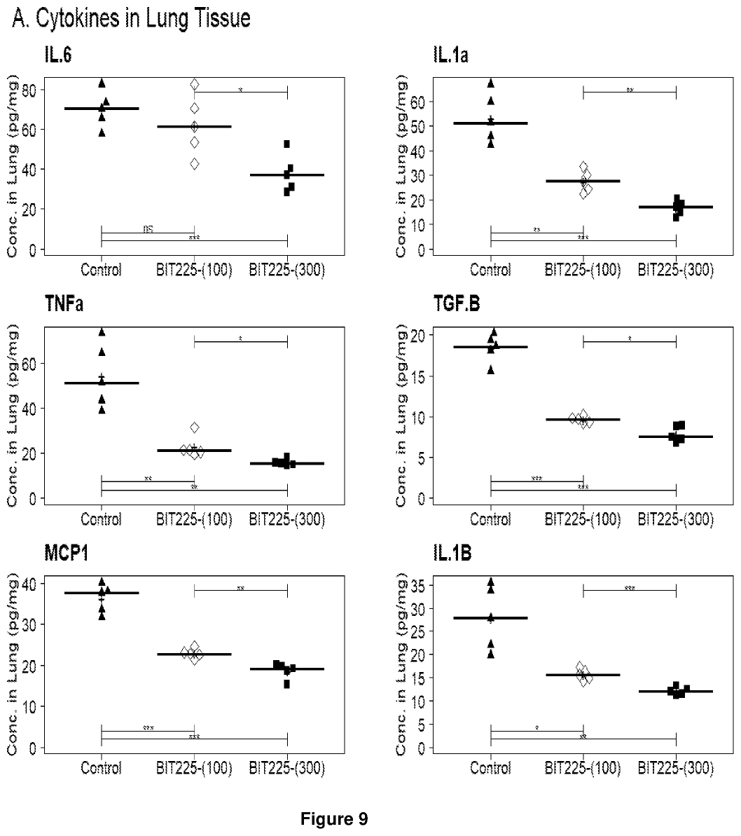

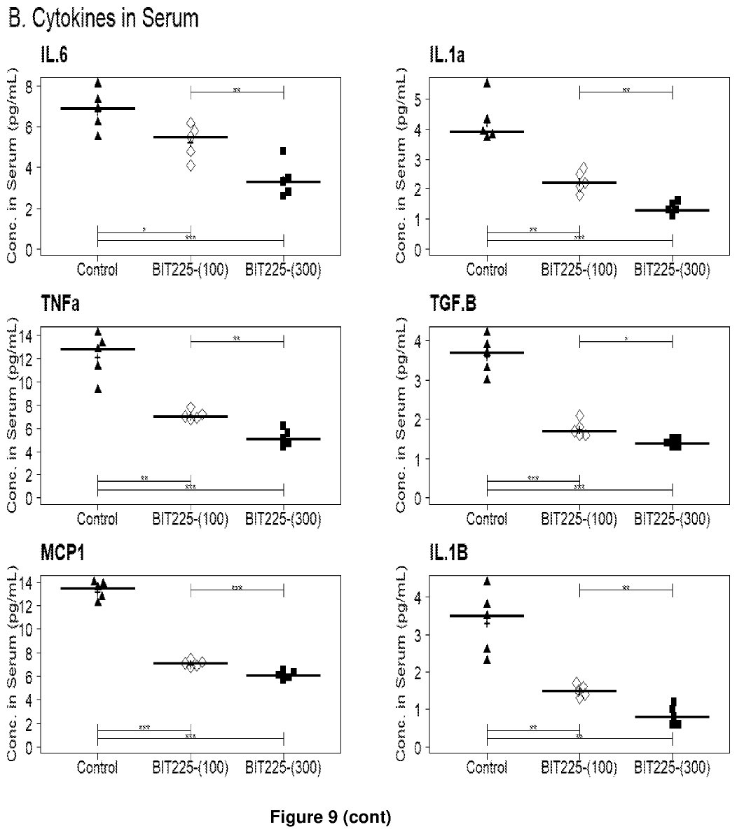

Consistent with reduced disease severity and spread of virus, mice dosed with BIT225 had reduced end-of-treatment levels of the 5 inflammatory cytokines (IL-6, IL-1a, IL-1B, TNFα & TGFβ) and one chemokine (MCP-1) measured in both lung and serum samples. With the exception of IL-6 in the 100 mg/kg dose group, all comparisons to the vehicle control yielded statistically significant (P<0.05) lower levels in the BIT225 groups. FIG. 9 shows the data for all fifteen mice measured 7 days post infection. Generally, mean and median cytokine concentrations in the high-dose BIT225 group were less than half the levels of the vehicle control group, and also lower than the low-dose group. A similar degree of cytokine reduction was measured in serum and lung samples from mice dosed for 5 and 12 days (not shown).

The in vivo results demonstrate that BIT225 inhibits SARS-COV-2 replication, reduces infectious viral load, reduces the production of exemplar pro-inflammatory cytokines and chemokines, and reduces the severity of complications associated with SARS-COV-2 infection.

1. A method for the treatment or prevention of SARS-COV-2 infection in a subject, the method comprising administering to the subject an effective amount of N-carbamimidoyl-5-(1-methyl-1H-pyrazol-4-yl)-2-naphthamide, or a pharmaceutically acceptable salt thereof.

2. The method of claim 1, wherein replication of SARS-COV-2 is inhibited.

3. The method of claim 1, wherein severity, intensity, or duration of complications or symptoms associated with SARS-COV-2 infection are reduced.

4. The method of claim 1, wherein the viral load is reduced.

5. The method of claim 1, wherein the production of proinflammatory cytokines or chemokines is reduced.

6. A method for the treatment or prevention of COVID-19 infection in a subject, the method comprising administering to the subject an effective amount of N-carbamimidoyl-5-(1-methyl-1H-pyrazol-4-yl)-2-naphthamide, or a pharmaceutically acceptable salt thereof.

7. The method of claim 6, wherein severity, intensity, or duration of complications or symptoms associated with COVID-19 are reduced.

8. The method of claim 1, wherein the N-carbamimidoyl-5-(1-methyl-1H-pyrazol-4-yl)-2-naphthamide, or a pharmaceutically acceptable salt thereof, is administered by a route selected from oral, nasal, intravenous, intraperitoneal, inhalation and topical.

9. The method of claim 1, wherein the N-carbamimidoyl-5-(1-methyl-1H-pyrazol-4-yl)-2-naphthamide, or a pharmaceutically acceptable salt thereof, is administered orally.

10. The method of claim 1, wherein the N-carbamimidoyl-5-(1-methyl-1H-pyrazol-4-yl)-2-naphthamide, or a pharmaceutically acceptable salt thereof, is administered at a dosage of about 100 mg to about 600 mg.

11. The method of claim 1, wherein the N-carbamimidoyl-5-(1-methyl-1H-pyrazol-4-yl)-2-naphthamide, or a pharmaceutically acceptable salt thereof, is administered orally, once daily at a dosage of about 100 mg to about 200 mg.

12. The method of claim 1, wherein the N-carbamimidoyl-5-(1-methyl-1H-pyrazol-4-yl)-2-naphthamide, or a pharmaceutically acceptable salt thereof, is administered orally, twice daily at a dosage of about 100 mg to about 200 mg.

13-19. (canceled)

20. The method of claim 6, wherein the N-carbamimidoyl-5-(1-methyl-1H-pyrazol-4-yl)-2-naphthamide, or a pharmaceutically acceptable salt thereof, is administered by a route selected from oral, nasal, intravenous, intraperitoneal, inhalation and topical.

21. The method of claim 6, wherein the N-carbamimidoyl-5-(1-methyl-1H-pyrazol-4-yl)-2-naphthamide, or a pharmaceutically acceptable salt thereof, is administered orally.

22. The method of claim 6, wherein the N-carbamimidoyl-5-(1-methyl-1H-pyrazol-4-yl)-2-naphthamide, or a pharmaceutically acceptable salt thereof, is administered at a dosage of about 100 mg to about 600 mg.

23. The method of claim 6, wherein the N-carbamimidoyl-5-(1-methyl-1H-pyrazol-4-yl)-2-naphthamide, or a pharmaceutically acceptable salt thereof, is administered orally, once daily at a dosage of about 100 mg to about 200 mg.

24. The method of claim 6, wherein the N-carbamimidoyl-5-(1-methyl-1H-pyrazol-4-yl)-2-naphthamide, or a pharmaceutically acceptable salt thereof, is administered orally, twice daily at a dosage of about 100 mg to about 200 mg.
